(19) United States
(12) Patent Application Publication
(10) Pub. No.: US 2016/0231342 A1
(43) Pub. Date: Aug. 11, 2016

(54) METHOD FOR PREPARATION OF PURIFIED ALLERGEN POSITIVE CONTROL SERUM

(71) Applicant: HOB BIOTECH GROUP SUZHOU CO., LTD., Suzhou, Jiangsu (CN)

(72) Inventor: Hong Wang, Suzhou, Jiangsu (CN)

(21) Appl. No.: 14/906,266

(22) PCT Filed: Nov. 14, 2014
(86) PCT No.: PCT/CN2014/091114

§ 371 Date: Jan. 20, 2016

(30) Foreign Application Priority Data
Oct. 17, 2014 (CN) 201410553626.5

(51) Int. Cl. G01N 33/96; G01N 33/531; B01D 15/38; G01N 33/68

(52) U.S. Cl. CPC G01N 33/96; G01N 33/6854; G01N 33/531; B01D 15/3809; G01N 2496/00; G01N 2800/24

(57) ABSTRACT

The present invention relates to a method for preparation of purified allergen positive serum. The method includes steps of using allergen to immune healthy animals, obtaining antiserum after blood sampling and purifying the antiserum for obtaining positive serum, the purification step including: affining and purifying the antiserum for obtaining IgG antibody; coupling the IgG antibody and human IgE Fc at a ratio of 1:1˜2 for obtaining IgG-IgE Fc conjugates concentrated solutions; separating and purifying the coupled IgG-IgE Fc conjugates concentrated solution; and diluting the IgG-IgE Fc conjugates concentrated solution to a concentration of 0.5˜1 μg/ml for obtaining the positive serum. The method of the present invention can batch preparation of a variety of allergen positive serum and can preparation of purified various other positive serum.

### FIELD OF THE INVENTION

The present invention is related to a technology for in vitro diagnostic, specifically on the preparation of the purified allergen positive control serum.

### BACKGROUND OF THE INVENTION

Allergy is an allergic reaction, or an abnormal response of human body to natural substances (allergens), which happens only in the susceptible population. There are hundreds of various allergens, including pollen, dust, proteins, chemicals, ultraviolet radiation, etc. While oxidation of free radicals in suppressed immune system of human body as an internal factor destroys mast cells and basophils, allergens as extrinsic factors play an important is role in the occurrence of allergic reactions. When an allergen enters the body for the first time, it activates mast cells or basophils to produce allergy mediating factors, such as leukotrienes, and prostaglandins. It is not an immediate allergic reaction, which phenomenon may last for two to three days, or even for several months. When the allergen re-enters the human body, mast cells deform and produce allergic factors, resulting in hypersensitive or allergic reactions.

Allergy refers to the hypersensitive reaction mediated by the immune system. Allergy can be humor (antibody) or cell-mediated immune response. In most cases, the reactions produce IgE antibodies, classified as IgE-mediated allergic reactions. However, not all specific reactions are

IgE-mediated. IgG is a factor that induce non-IgE-mediated allergic reactions, such as the anaphylactic shock caused by dextran-containing immune complexes, classified as the type III allergic reaction. In patients with allergic bronchopulmonary aspergillosis (ABPA), both IgE and IgG antibodies can be detected. Contact allergic dermatitis represents lymphocyte-mediated allergic diseases.

Currently, due to the registration concern for skin prick test while in vitro testing experiences significant technology advancement, in vitro testing has become the main stream method for allergy diagnosis. However, the methodology for serologic allergen specific IgE detection has not been improved much in the past years, thus a high quality and economic product becomes the main request from the in vitro diagnostic market.

For the development of allergy IVD, the positive control serum is an important tool, but the availability of positive sera for many allergen-specific allergies is very limited, if any. Approaches such as artificial sera have been attempted. For example, the patent CN103018436A published on Apr. 3, 2013, disclosed a method for preparing Infectious Bronchitis positive serum in rabbits. The whole blood from the immunized rabbit was centrifuged and filtered, and resulted in the positive serum. The crude preparation may result in low titers, which makes the artificial sera less valuable in the assay development.

### SUMMARY OF THE INVENTION

The present invention is to provide a technical solution through a purification method in preparing high titer allergen positive sera.

The description of this method is as below:

A method for preparation of purified allergen positive serum, wherein the method comprises steps of using allergen to immune healthy animals, obtaining antiserum after blood sampling and purifying the antiserum for obtaining positive serum, the purification step comprising:
affining and purifying the antiserum for obtaining IgG antibody;
coupling the IgG antibody and human IgE Fc at a ratio of 1:1˜2 for obtaining IgG-IgE Fc conjugates concentrated solutions;
separating and purifying the coupled IgG-IgE Fc conjugates concentrated solution; and
diluting the IgG-IgE Fc conjugates concentrated solution to a concentration of 0.5˜1 μg/ml for obtaining the positive serum.

Optimized, the antiserum is affined and purified by agarose affinity media, or immune affinity chromatography column.

Optimized, the agarose affinity medium is Protein-A sepharose CL-4B.

Optimized, the affinity chromatography column is prepared by coupling the allergens to the sepharose gel.

Optimized, the antiserum is affined and purified by the immune affinity chromatography column after it is disposed by Ammonium sulfate.

Optimized, the human IgE Fc is prepared by the following steps:
firstly, dissolving human IgE into papain digestive juices;
secondly, using the papain to digest the human IgE with digestion reaction;
thirdly, using iodoacetamide to terminate the digestion reaction;
finally, extracting the human IgE Fc through agarose affinity media.

Optimized, the IgG antibody and the human IgE Fc in the IgG-IgE Fc conjugates is firstly activated by 2-imine tetrahydrothiophene coupling agent or 4-(N-maleic imide methyl) cyclohexane-1-carboxylic acid succinimide ester coupling agent, and then coupled in condition of pH 7.2˜7.4.

Optimized, a concentration of 2-imine tetrahydrothiophene coupling agent is 9˜11 mg/ml, a concentration of 4-(N-maleic imide methyl) cyclohexane-1-carboxylic acid succinimide ester coupling agent is 4˜6 mg/ml.

Optimized, the coupled IgG-IgE Fc conjugates concentrated solution is separated and purified using Sephadex 200 Gel purification column.

Optimized, the IgG-IgE Fc conjugates concentrated solution is diluted using a dilution buffer containing BSA 0.4˜0.6%, Tris buffer 0.09˜0.11 mol/L, and PH 7.5˜8.5.

Due to the above described technical advancement, the present invention has the following advantages compared to the existing technologies:

The production can be scaled up (from a few milliliters to several thousand milliliters) to suite the need, and effectively maintain the lot-to-lot consistency, which is unattainable with the crude positive serum preparation methods. In addition, the IgE Fc fragment is utilized to conjugate onto allergen specific IgG from the anti-serum, which conjugate molecule IgG-IgE Fc is much smaller than IgG-IgE, consequently decrease the cross reactivity and increase the specificity, and preserve the secondary antibody binding sites to a great extent.

The said method can be easily implemented to prepare positive sera for a variety of allergens. It solves the availability of positive sera in assay development and reagent manufacture, including calibrator preparation.

### BRIEF DESCRIPTION OF THE DRAWINGS

FIG. 1 shows the process of the purification of the rabbit anti-willow IgG-IgE Fc conjugate.

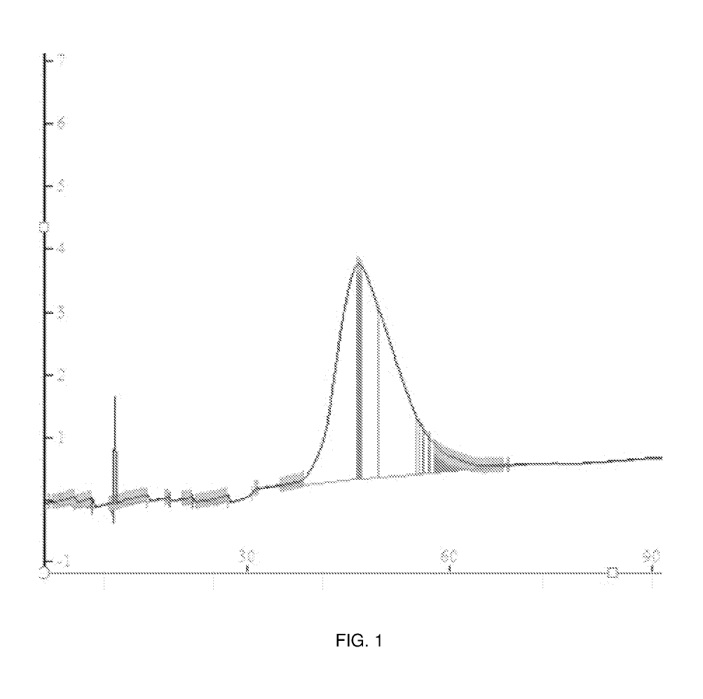

### DETAILED DESCRIPTION OF EMBODIMENTS OF THE INVENTION

The present invention will be further described in detail in conjunction with the specific embodiments below, but the present invention is not limited to the following examples. Conditions used in the example embodiments may be further adjusted according to different requirements of a particular use. Conditions that are not indicated in the example embodiments are common conditions.

### Example 1

### First: Rabbit Allergen Immunization and Anti-Serum Titering

### Materials and Equipment:

1. Allergen: lyophilized beef allergen.

2. Adjuvant: Freund's complete adjuvant, Sigma (F5881); Freund's incomplete adjuvant, Sigma(F5506).

3. Animals: Three (for each allergen), two-month old, 1.5-2.0 kg, healthy New Zealand rabbits.

4. The HRP-conjugated secondary antibody: HRP-conjugated goat anti-rabbit IgG.

5. Consumables: tee, disposable syringe, pipettes, etc. Immunization procedure:

1. Allergen preparation: Dissolve the allergen with PBS (to ensure the final conc. ranging 4-8 mg/ml), and measure protein concentration using the BCA protein quantification kit. Dilute 4 mg allergen to 600 μl, mix with complete or incomplete adjuvant (allergen V:Adjuvant V =6:5). Emulsify the mixture by pushing through the tee, till the drop into water is spherical without diffusion. Use Freund's complete adjuvant in the first immunization and Freund's incomplete adjuvant in the subsequent immunizations.

2. Animal Immunization: Keep the three newly purchased New

Zealand rabbits in the animal room for one week; inject the allergen-adjuvant mixture subcutaneously in neck or back using the 1 ml syringe; each rabbit receives injection at 3˜4 points, with 200-350 μl per point; the immunization interval is 14 days.

3. Antiserum preparation: Draw 2 ml blood from each animal through the ear vein prior to immunization (blank), and once every 14 days post-immunization (Sample). Transfer the blood from the syringe slowly into a centrifuge tube and store overnight at 4° C., with the supernatant appearing yellowish. The antiserum is then transferred to another tube, centrifuge for 10 min at 1,500 xg and combine the supernatants as antiserum. Aliquot and store the antiserum at −70° C.

4. Antisera is titered by ELISA. Allergen is diluted in the coating buffer to 10 μg/ml. In each ELISA microplate, first two wells are coated with irrelevant proteins, 1 μg mutton proteins and 1 μg peanut proteins, respectively, as negative controls. The rest of wells are coated with 100 μl, 1 μg allergen (beef). Incubate for overnight at 4° C. After coating and washing, add 1:100 diluted rabbit anti-serum into the negative wells, followed with 3 blank control wells (Blank 1: serum prior to allergen injection; Blank 2: primary antibody blocking buffer; Blank 3: secondary antibody blocking buffer), and then serial dilution of rabbit anti-serum in the rest of wells. Finally, the enzyme-labeled goat anti-rabbit IgG antibody is added to determine the titer of rabbit anti-serum.

5. Each subsequent titer is measured against the one from the injection immediately preceding. If the two titers are insignificantly different, the animals are sacrificed to collect the blood. Table 1 shows the results of titers of rabbit antisera post beef allergen immunization.

TABLE 1

| | A | B |
|---|---|---|
| Sample | OD value | OD value |
| Negative Control | 0.066 | 0.076 |
| One | | |
| Negative Control | 0.071 | 0.069 |
| Two | | |
| Blank Control One | 0.078 | 0.072 |
| Blank Control Two | 0.067 | 0.077 |
| Blank Control Three | 0.074 | 0.068 |
| 1:100 | 3.312 | 3.265 |
| 1:200 | 3.297 | 3.271 |
| 1:400 | 3.054 | 3.142 |
| 1:800 | 2.993 | 2.879 |
| 1:1,600 | 2.853 | 2.775 |
| 1:3,200 | 1.631 | 1.645 |
| 1:6,400 | 0.983 | 0.932 |
| 1:12,800 | 0.552 | 0.571 |
| 1:25,600 | 0.312 | 0.334 |
| 1:51,200 | 0.184 | 0.169 |
| 1:102,400 | 0.0107 | 0.073 |
| 1:200,000 | 0.073 | 0.062 |
| 1:400,000 | 0.069 | 0.071 |
| 1:800,000 | 0.072 | 0.065 |
| 1:1,600,000 | 0.065 | 0.073 |

Note:
A, titer post the 6^{th} injection;
B, titer post the 7^{th} injection.

Above data show that the titers between post 6^{th} and 7^{th} injection are very similar and appear to be stable. The positive results have OD values twice that of the blank, such as the 1:51,200 dilution, while the negative results have OD values less than twice that of the blank. Negative controls have OD values equivalent to those of the blanks, confirming that this antiserum is not reacting with irrelevant allergens. Blanks have low OD values confirming that the beef allergen has no non-specific reaction with pre-immunization rabbit serum or the buffers. The reaction from the rest of the wellsis specific between beef allergen and rabbit anti-beef allergen sera, with OD values correlate with the dilution factor.

### Second: Affinity Purification of the Rabbit Anti-Allergen Polyclonal Antibody Materials and Equipment:

1. Protein A sepharose CL-4B; peristaltic pump; centrifuge tube; centrifuge; filter; glass column.

2. TBS buffer: 6.06 g Tris (50 mM), 8.78 g NaCl (150 mM) and 0.5 g sodium azide (0.05%) are dissolved in 1 L distilled water; adjust to pH7.4 with HCl.

3. Neutralization buffer: 121.2 g Tris (1 M), 87.8 g NaCl (1.5 M), 0.37 g EDTA (1 mM) and 5 g sodium azide (0.5%) are dissolved in 1 L distilled water; adjust to pH8.0 with HCl.

4. Elution buffer (pH 2.7): Dissolve 3.75 g glycine (50 mM) in 1 L distilled water; adjust to pH 2.7 with HCl.

5. Elution buffer (pH 1.9): Dissolve 3.75 g glycine (50 mM) in 1 L distilled water; adjust to pH 1.9 with HCl.

### Procedure:

1. Mix equal volumes of resin and TBS buffer in a vacuum flask. Mix under vacuum for about 15 minutes to remove air bubbles in the resin. The protein A sepharose CL-4B resin is slowly transferred into the glass column, control the fill rate of 1-2 ml min with a pump, then equilibrate the column using ten times the bed volume of ice-cold TBS buffer. 2. Place the antiserum at 4° C. to thaw slowly to avoid the protein aggregation. If aggregation occurs in the thawing process, place the tube at 37° C. to warm-up and dissolve aggregates. Sodium azide is added to 0.05%, centrifuge at 4° C. 15,000 xg for 5 minutes, transfer the supernatant and filtrate to remove excess fat.

3. Dilute the thawed antiserum at 1:5 (v/v) with TBS buffer, and then filtrate. Add the antiserum twice onto the column at a rate of 0.5 ml per minute, and collect the flow through. Wash the column with TBS buffer till A₂₈₀ₙₘ<0.008. The bound protein is eluted by the elution buffer(pH2.7) at 0.5 ml/min. Collect the eluent into 1.5 ml EP tube which has 100 μl neutralize buffer solution. Mix and check the eluent pH with pH test paper. If the pH<7, adjust to about pH7.4 with neutralize buffer solution to prevent antibody degeneration.

4. Add 10 ml, pH1.9 elution buffer in the column, and collect the eluate until A₂₈₀ₙₘ<0.008 as described above.

5. Measure the protein concentration with a spectrophotometer. If the protein concentration is less than 0.5 mg/ml, add 10% glycerol. Aliquot and store the purified antibody at 2-8° C.

6. Wash the column with TBS-0.05% NaN3, and store the column at 2-8° C.

Third: Conjugation of IgG with human IgE Fc

### Materials and Equipment

1. The human IgE Fc, produced in HOB Biotech Group Co. Ltd, stored in phosphate buffer.

2. Papain Buffer: 0.1 M Tris, 2 mM EDTA, pH8.0.

3. Papain and lodoacetamide, Sigma; Protein-A, GE.

4. SMCC and Traut's reagent, Thermo; TRIS and other chemical reagents, chemical purity.

5. G-25 gel column and Supperdex 200gel purification column, GE.

### Procedure

1. Dissolve the human IgE into Papain buffer, add Papain to start and lodoacetamide to stop the reaction, then purify human IgE Fc with protein A purification column;

2. Transfer 1 mg rabbit-anti IgG, add 3 μl 10 mg/ml coupling reagent2-ITsolution, incubateat room temperature for 20 min; add 10μl 0.1 mol/L glycine solution, incubate at room temperature for 5 min. Desalt with G-25gelcolumn, collect the activated antibody, and store at 5° C.;

3. Transfer 1.5 mg human IgE Fc,add 15 μl 5 mg/ml SMCC, incubate at room temperature for 30 min. Desalt with G-25 gel column, collect the activated antibody, store at 5° C.;

4. Mix the above-activated IgG antibody and human IgE Fc, react at pH 7.3 for 20 h; purify the conjugate with Sephacryl-200 gel purification column, and store at 5° C.;

5. Dilute the IgG-IgE conjugate stock solution with 0.1 mol/L pH 8.0 Tris buffer (containing 0.5% BSA) to 0.5 μg/ml.

Fourth: Use the specific allergen IgE antibody assay kit from Phadia to measure the activity of IgG-IgE Fc conjugate. The results are shown in Table 2.

TABLE 2

| | Dilution | |
|---|---|---|
| Name | factor | Phadia Results |
| IgG-IgE FC (F27) | Neat | 73.2 kU/L |
| LOT131217 | 1:3 | 26.9 kU/L |
| | 1:9 | 10.3 kU/L |
| IgG-IgE FC (F27) | Neat | 83.5 kU/L |
| LOT140319 | 1:3 | 33.9 kU/L |
| | 1:9 | 15.6 kU/L |
| IgG-IgE FC (F27) | Neat | 69.7 kU/L |
| LOT140608 | 1:3 | 26.8 kU/L |
| | 1:9 | 11.9 kU/L |

Note:
Phadia's score system for allergen-specific IgE:
<0.35 kU/L, negative;
0.35-0.69 kU/L, positive Class I;
0.7-3.49 kU/L, positive Class II;
3.5-17.49 KU/L, positive Class III;
17.5-49.99 kU/L, positive Class IV;
50-99.99 kU/L, positive Class V;
>100 kU/L, positive Class VI.

Data in Table 2 show that the three batches (LOT131217, LOT140319, LOT140608) of positive serum control samples are Class V positive samples. The values are linearly correlated with the dilution factors. The preparation of IgG-IgE Fc conjugate as allergen-specific antisera have been successful and the samples can be used as controls for the assay kit.

Example 2

First, the titration of rabbit anti-allergen antisera

### Material and Equipment

1. Allergens: lyophilizedwillow allergen.

2. Adjuvant: Freund's complete adjuvant, Sigma (F5881); Freund's incomplete adjuvant, Sigma (F5506).

3. Animals: Three (for each allergen), two-month old, 1.5 -2.0 kg, healthy New Zealand rabbits.

4. The HRP-conjugated secondary antibody: HRP-conjugated goat anti-rabbit IgG.

5. Consumables: tee, disposable syringe, pipettes, etc.

Immune procedure

1. Immune allergen preparation: Dissolve the allergen with PBS and measure protein concentration using the BCA protein quantification kit. For the first immunization, dilute the allergen 0.05 mg, 0.15 mg and 0.2 mg in 300 pL PBS, and mix with freund's complete adjuvant as 0.8:1 (v/v) by pipetting up and down for 10 times and use immediately. For the second immunization and on, dilute allergen to 0.05 mg, 0.15 mg and 0.2 mg in 500 μL PBS, and mix with freund's incomplete adjuvant at a ratio of 0.8:1 (v/v) by pipetting up and down for about 10 times and use immediately.

2. Animal immunization: Keep the three white New Zealand rabbits in the animal room for 1 week. In the first immunization, inject 0.5 m1 mixture using a 1 ml syringe to the rabbit's foot. Later immunizations are performed through leg and breast muscle injection. The immunization interval is 7 days.

3. Antiserum preparation: Collect2 ml blood from each animal through the ear vein prior to immunization (control), and once every two weeks post immunization (sample). When the titer meets requirements, collect large volume of blood through heart blood collection. The blood is centrifuged at 1,000 rpm for 15 min. Aliquot and store the antiserum at −70° C.

4. Antiserum titer determination: Use the microplate chemiluminescence method to evaluate the antiserum. Allergen is diluted in the coating buffer to 5 μg/ml. Add 100 μL,0.5 μg allergens per well except the negative control wells, and incubate at 37° C. for 2 hours. The primary antibody is the serially diluted antiserum, and the secondary antibody is the sheep anti-rabbit IgG conjugated to AP. The negative control: serially diluted pre-immunization antiserum. Blank: use the blocking buffer. The antisera are serially diluted as 1:200, 1:400 till 1:100,000, meeting criteria.

The results in Tables 3 and 4 show the titering results for the rabbit anti-willow-sera post immunization.

TABLE 3

| | Animal Serial No. |
|---|---|
| Serum Serial | 672 |

| No. | Positive | Negative |
|---|---|---|
| Blank | 32104 | |
| comparison | | |
| 1:200 | 8387823 | 97814 |
| 1:400 | 6626126 | 36807 |
| 1:800 | 5436566 | 32310 |
| 1:1600 | 4176213 | 28020 |
| 1:3200 | 2896923 | 28651 |
| 1:6400 | 1998789 | 26093 |
| 1:12800 | 1250476 | 28367 |
| 1:25600 | 715606 | 30144 |
| 1:51200 | 367424 | |
| 1:102400 | 200716 | |
| 1:204800 | 111176 | |
| 1:409600 | 67601 | |
| 1:800000 | 56854 | |
| 1:1600000 | 40704 | |
| 1:3200000 | 35891 | |

TABLE 4

| | Animal Serial No. | | | |
|---|---|---|---|---|
| | 671 | | 670 | |
| Serum Serial | | Positive | | Negative |
| No. | Positive | Comparison | Negative | Comparison |
| Blank | 26837 | | 27764 | |
| comparison | | | | |
| 1:200 | 7523439 | 137451 | 7504500 | 143790 |
| 1:400 | 6352811 | 69286 | 6204636 | 71080 |
| 1:800 | 5599561 | 62931 | 5429980 | 66423 |
| 1:1600 | 4545014 | 54381 | 4263473 | 50904 |
| 1:3200 | 3416377 | 44317 | 3248440 | 39157 |
| 1:6400 | 2549519 | 33341 | 2453204 | 32216 |
| 1:12800 | 1847314 | 29854 | 1730413 | 30516 |
| 1:25600 | 1326213 | 34961 | 1212607 | 29911 |
| 1:51200 | 633817 | | 679976 | |
| 1:102400 | 334799 | | 318177 | |
| 1:204800 | 201637 | | 184946 | |
| 1:409600 | 114124 | | 108490 | |
| 1:800000 | 69803 | | 67627 | |
| 1:1600000 | 51583 | | 50146 | |
| 1:3200000 | 39241 | | 40323 | |

TABLE 5

| | Animal Serial No. | | |
|---|---|---|---|
| | 672 | 671 | 670 |
| Antiserum titer | 1:100000 | 1:200000 | 1:100000 |

Note:
The antiserum titers are from 3 rabbits post the 4^{th} injection;

Above data show that the titer of the antisera post the 4^{th} injection meet the requirements and the tiers are greater than 1:100,000. According to the experimental design, the serum dilution ranges from1:200 to 1:3200000.When RLU is above twice that of the negative control at 1:200 dilution (criteria for positive), the result is positive, otherwise negative. Rabbits 672, 671 and 670 antisera at 1:100,000 dilution, all have RLU above twice of the control, so all positive. Further dilution results in RLU below criteria, so becomes negative.

### Second, Antiserum Affinity Purification

### Material and Equipment:

1. Equipment and supply: Immune affinity chromatography column, peristaltic pump and centrifugal pipe, centrifuge, filter, column chromatography, spectrophotometer;

2. TBS buffer: Dissolve 6.06 g Tris (50 mM), 8.78 g NaCl (150 mM), and 0.5 g sodium azide (0.05%) in 1L distilled water, and adjust to pH 7.4 with HCl;

3. High salt buffer solution: Dissolve 121.2 g Tris (1 M), 87.8 g NaCl (1.5 M), EDTA (1 mM) 0.37 g and 5 g sodium azide (0.5%) in 1 L distilled water, and adjust to Ph8.0 with HCl;

4. Elution buffer: Dissolve 3.75 g glycine (50 mM) in 1L distilled water, and adjust to pH 2.6 with HCl;

5. Other materials: 0.2 M carbonate buffer (pH 9.5), 1.0 mM HCl, 1.0 M glycine solution, CNBr-actived Sepharose 4B-Cl(GE), column materials storage solution (0.1 M PBS, 1% amino caproic acid, pH 7.4).

Operation Procedure 1. Immuno-affinity chromatography column: Couple the antigen onto the agarose gel to obtain the affinity chromatography column. Specific procedures are as following:
Dilute the specific allergen to 1.0 mg/ml with 0.2M carbonate buffer (pH 9.5);
Treat the activated Sepharose 4B-Cl (CNBr-active Sepharose 4B-Cl) with 1.0 mM HCL, and dilute to 1.0 g/ml in the 0.2 M carbonate buffer (pH 9.5);
Mix the two parts at 1:1, and react at room temperature for 16 to 20 hours, centrifuge and collect the supernatant;
Measure protein concentration of the supernatant, subtract from the total to derive the amount of antigen coupled to the resin in the column.
Add the remaining Sepharose4B-Cl into 1.0 M glycine solution at 1:1 ratio, react at room temperature for 4 hours. Wash the gel with 0.1 M HCl, 0.1 M NaOH and 2 M urea sequentially with 3 times column bed volume. Store the resin in the storage buffer.

2. Mix well at equivalent volumes the affinity chromatography resin and animal serum processed with the ammonium persulfate for 2 hours. Transfer the mixture slowly into the glass column, and control the filling rate at 1-2 ml/min with a pump and avoid the geldrying. Apply 3-10 bed volume of buffer to equilibrate the column.

3. Wash the column with TBS buffer till A_{280 nm}<0.008. Then apply the high salt buffer to remove the nonspecifically bound proteins. The bound protein is eluted with the elution buffer(pH2.6) at 0.5 ml/min. Collect the eluent into 1.5 ml EP tubes prefilled with 100 μl neutralize buffer till A_{280 nm}<0.008. Mix and check the eluent pH with pH test paper, if the pH<7, adjust to about pH7.4 with neutralization buffer to avoid antibody degeneration.

4. Measure the protein concentration with a spectrophotometer. If the protein concentration is less than 0.5 mg/ml, add 10% glycerol. Aliquot and store the purified antibody at 2-8° C.

5. Wash the column with TBS(0.05%NaN3 added), and store the column at 2-8° C.

### Third: IgG-IgE Fc Conjugate

### Materials and Equipment

1. The human IgE Fc, produced in HOB Biotech Group Co. Ltd, stored in phosphate buffer.

2. Papain Buffer: 0.1 M Tris, 2mM EDTA, pH8.0.

3. Papain and Iodoacetamide, Sigma; Protein-A, GE.

4. SMCC and Traut's reagent, Thermo; TRIS and other chemical reagents, chemical purity. 5. G-25 gel column and Supperdex 200 gel purification column, GE.

### Operation Procedure

1. Dialyze human IgE is with 95% purity,1 mg/ml into Papain Buffer (pH 8.0). Add Papain at a ratio of Papain: IgE=100:1 (w/w), and incubate for 30 min. Then add Iodoacetamide to stop the reaction. Purify human IgE Fc with protein A purification column;

2. Add 15 μL of 5 mg/ml SMCC solution to 1 mg IgG antibody solution, and incubate at room temperature for 30 min. Use G-25 gel column to remove the free SMCC, and collect activated IgG, and store at 4° C.;

3. Mix 1.5 mg human IgE Fc with 3 μL 10 mg/ml coupling agent 2-IT solution, and incubate at room temperature for 20 min. Then add 10 μL 0.1 mol/L glycine solution, and incubate at room temperature for 5 min. Use G-25 gel column to remove the free 2-IT, and collect antibodies after activation, and store at 4° C.;

4. Mix the said activated IgG antibody and human IgE Fc, react at pH 7.3 for 20 h; purify the conjugate with Sephacryl-200 gel purification column, and store at 4° C.

5. Dilute the IgG-IgE conjugate stock solution to 0.5 μg/ml with 0.1 mol/L Tris buffer (with0.5% BSA, pH 8.0).

Fourth, use specific allergen IgE antibody test kits manufactured by Phadia to detect the positive serum in example 2. The results are shown in Table 6.

TABLE 6

| | Dilution | Phadia | |
|---|---|---|---|
| name | factor | Results | |
| IgG-IgE FC | Neat | >100 | kU/L |
| (T12) | 1:3 | 57.2 | kU/L |
| LOT140105 | 1:9 | 20.03 | kU/L |
| IgG-IgE FC | Neat | 97 | kU/L |
| (T12) | 1:3 | 35 | kU/L |
| LOT140327 | 1:9 | 12.5 | kU/L |
| IgG-IgE FC | Neat | >100 | kU/L |
| (T12) | 1:3 | 77.9 | kU/L |
| LOT140507 | 1:9 | 25.53 | kU/L |

Note:
Phadia's score system for allergen-specific IgE:
<0.35 kU/L, negative;
0.35-0.69 kU/L, positive Class I;
0.7-3.49 kU/L, positive Class II;
3.5-17.49 kU/L, positive Class III;
17.5-49.99 kU/L, positive Class IV;
50-99.99 kU/L, positive Class V;
>100 kU/L, positive Class VI.

It can be seen from Table 6 that the three batches (LOT140105; LOT140327; LOT140507) positive serum control samples are Class V positive samples. The values are linearly correlated with the dilution factors. The preparation of IgG-IgE Fc conjugate as allergen-specific antisera have been successful and the samples can be used as controls for the assay kit.

A detailed description of the invention is presented above to make the content and implementation of the present invention understandable to the field, which should not place any limitation on the extent of protection of the present invention. All the spiritual essence of the equivalent change and modification according to the invention should be within the scope of protection of the present invention.

1. A method for preparation of purified allergen positive serum, the method comprising steps of using allergen to immune healthy animals, obtaining antiserum after blood sampling and purifying the antiserum for obtaining positive serum, wherein the step of purifying the antiserum for obtaining positive serum are as follows:
affining and purifying the antiserum for obtaining IgG antibody;
coupling the IgG antibody and human IgE Fc at a ratio of 1:1˜2 for obtaining IgG-IgE Fc conjugates concentrated solutions;
separating and purifying the coupled IgG-IgE Fc conjugates concentrated solution; and
diluting the IgG-IgE Fc conjugates concentrated solution to a concentration of 0.5˜1 μg/ml for obtaining the positive serum.

2. The method of claim 1, wherein the antiserum is affined and purified by agarose affinity media, or immune affinity chromatography column.

3. The method of claim 2, wherein the agarose affinity media is Protein-A sepharose CL-4B.

4. The method of claim 2, wherein the affinity chromatography column is prepared by coupling the allergens to the sepharose gel.

5. The method of claim 2, wherein the antiserum is affined and purified by the immune affinity chromatography column after it is disposed by Ammonium sulfate.

6. The method of claim 1, wherein the human IgE Fc is prepared by the following steps:
firstly, dissolving human IgE into papain digestive juices;
secondly, using the papain to digest the human IgE with digestion reaction;
thirdly, using iodoacetamide to terminate the digestion reaction;
finally, extracting the human IgE Fc through agarose affinity media.

7. The method of claim 1, wherein the IgG antibody and the human IgE Fc in the IgG-IgE Fc conjugate is activated by 2-imine tetrahydrothiophene or 4-(N-maleic imide methyl) cyclohexane-1-carboxylic acid succinimide ester, respectively, and coupled at pH 7.2-7.4.

8. The method of claim 7, wherein a concentration of 2-imine tetrahydrothiophene is 9-11 mg/ml, and a concentration of 4-(N-maleic imide methyl) cyclohexane-1-carboxylic acid succinimide ester is 4-6 mg/ml.

9. The method of claim 1, wherein the IgG-IgE Fc conjugate is purified using the Sephadex 200 Gel purification column.

10. The method of claim 1, wherein the IgG-IgE Fc conjugate is diluted using a buffer containing 0.09-0.11 mol/L Tris, 0.4-0.6% BSA, pH 7.5-8.5.

11. The method of claim 4, wherein the antiserum is affined and purified by the immune affinity chromatography column after it is disposed by Ammonium sulfate.
